(19) United States
(12) Patent Application Publication
(10) Pub. No.: US 2025/0164811 A1
(43) Pub. Date: May 22, 2025

(54) INCLINATION DETECTOR, LINE-OF-SIGHT DETECTOR, HEAD-MOUNTED DISPLAY, RETINAL PROJECTION DISPLAY, EYE TEST DEVICE, USER STATE ESTIMATION DEVICE, AND DRIVING SUPPORT SYSTEM

(71) Applicants: Suguru SANGU, Kanagawa (JP); Saori YOSHIDA, Tokyo (JP); Naoto JIKUTANI, Miyagi (JP); Junichi KONISHI, Hyogo (JP)

(72) Inventors: Suguru SANGU, Kanagawa (JP); Saori YOSHIDA, Tokyo (JP); Naoto JIKUTANI, Miyagi (JP); Junichi KONISHI, Hyogo (JP)

(21) Appl. No.: 18/839,044

(22) PCT Filed: Mar. 13, 2023
(86) PCT No.: PCT/IB2023/052375

§ 371 Date: Aug. 16, 2024

(30) Foreign Application Priority Data
Mar. 18, 2022 (JP) 2022-044644

(51) Int. Cl. G02B 27/01

(52) U.S. Cl. CPC G02B 27/0179; G02B 2027/0187

(57) ABSTRACT

An inclination detector includes: a first support including: a first surface; and a second surface opposite to the first surface; a light source on the first surface, the light source to emit a light beam; a light guide to guide the light beam emitted from the light source; a light exit portion to emit the light beam, guided through the light guide, from the first surface toward the second surface; and a first light receiver on the second surface of the first support, the first light receiver to receive the light beam emitted from the light exit portion and reflected from a three-dimensional object and output a first reception signal.

The present disclosure relates to an inclination detector, a line-of-sight detector, a head-mounted display, a retinal projection display, an eye test device, a user state estimation device, and a driving support system.

### BACKGROUND ART

A device to detect the inclination of a three-dimensional object such as a human eyeball is known. Such an inclination detector (an inclination detection device) is used in a line-of-sight detector, a head-mounted display, a retinal projection display, or an eye test device.

For example, PTL 1 discloses an optical information reading device that optically scans a reading object having patterns of different reflectance and receives the reflected light of the reading object, and reads information from a signal obtained by photoelectric conversion. The optical information reading device includes: a substrate including: a light emitting portion; and a scanning mirror to deflect the light emitted from the light emitting portion; a reflector unit including a reflection surface to guide the light emitted from the light emitter to the scanning mirror; and a collimator unit to collimate the light guided to the scanning mirror into parallel light. The substrate, the reflector unit, and the collimator unit are formed on a wafer to bond.

However, in the configuration of the optical information reading device in PLT1, there is room to reduce the size of the device.

### CITATION LIST

### Patent Literature

### [PTL 1]

Japanese Patent No. 4914616

### Non Patent Literature

### [NPL 1]

Ohsima S Optical Mechanism of the Eye. Journal of the Japan Society of Precision Engineering, volume 27 issue 322 750-755, 1961.

### [NPL 2]

Tseng, V. WS, Valliappan, N., Ramachandran, V. et al. Digital biomarker of mental fatigue. NPJ Digit. Med. 4, 47, 2021.

### [NPL 3]

Pastukhov A, Braun J. Rare but precious: microsaccades are highly informative about attentional allocation. Vision Res. 2010 Jun. 11; 50 (12): 1173-84.

### SUMMARY OF INVENTION

### Technical Problem

An aim of the present invention is to provide an inclination detector that can reduce the size.

### Solution to Problem

An inclination detector includes: a first support including: a first surface; and a second surface opposite to the first surface; a light source on the first surface, the light source to emit a light beam; a light guide facing the light source, the light guide to guide the light beam emitted from the light source; a light exit portion to emit the light beam through the first support, the light beam guided from the light source on the first surface toward the second surface through the light guide; a first light receiver on the second surface of the first support, the first light receiver to receive the light beam emitted from the light exit portion and reflected from a three-dimensional object and output a first reception signal; and circuitry to detect an inclination of the three-dimensional object based on the first reception signal output from the first light receiver.

Further, an embodiment of the present disclosure provides a line-of-sight detector including the inclination detector described above. The three-dimensional object includes an eyeball, and the line-of-sight detector detects a line-of-sight of the eyeball.

Further, an embodiment of the present disclosure provides a head-mounted display including the line-of-sight detector described above.

Further, an embodiment of the present disclosure provides a retinal projection display including the line-of-sight detector described above.

Further, an embodiment of the present disclosure provides an eye test device including the line-of-sight detector described above.

Further, an embodiment of the present disclosure provides a user state estimation device including: the line-of-sight detector described above; and an estimation unit to estimate a state of the user based on the line-of-sight of the eyeball.

Further, an embodiment of the present disclosure provides a driving support system including: the user state estimation device described above; and circuitry to control a mobile body driven by the user based on the state of the user estimated by the user state estimation device.

### Advantageous Effects of Invention

According to the present the embodiments, an inclination detector that can reduce the size is provided.

### BRIEF DESCRIPTION OF DRAWINGS

The accompanying drawings are intended to depict example embodiments of the present invention and should not be interpreted to limit the scope thereof. The accompanying drawings are not to be considered as drawn to scale unless explicitly noted. Also, identical or similar reference numerals designate identical or similar components throughout the several views.

FIG. 1 is a diagram of an overall configuration of a line-of-sight detector according to an embodiment;

FIG. 2 is a bottom view of an optical unit according to the embodiment;

FIG. 3 is a cross-sectional view of the optical unit taken along the line III-III in FIG. 2;

FIG. 4 is a top view of the optical unit according to the embodiment;

FIG. 5 is a top view of a first substrate in the optical unit according to the embodiment;

FIG. 6 is a block diagram of a hardware of circuitry according to the embodiment;

FIG. 7 is a block diagram of a functional configuration of the circuitry according to the embodiment;

FIG. 8 is a diagram of a configuration of a light source according to a first embodiment;

FIG. 9 is a cross-sectional view of the light source taken along a line IX-IX in FIG. 8;

FIG. 10 is a diagram of a configuration of a light source according to a second embodiment;

FIG. 11 is a cross-sectional view of the light source taken along the line XI-XI in FIG. 10;

FIG. 12 is a diagram of a configuration of a line-of-sight detector according to a third embodiment;

FIG. 13 is a diagram of a configuration of a retinal projection display according to a fourth embodiment.

### DESCRIPTION OF EMBODIMENTS

Embodiments of the present disclosure will be described with reference to the drawings. In the drawings, the same components are denoted by the same reference number, and redundant description thereof will be appropriately omitted. The embodiments described below are examples of an inclination detector for embodying the technical idea of the present disclosure, and the present disclosure is not limited to the embodiments described below. The shapes of components, relative arrangements thereof, values of parameters, and the like described below are not intended to limit the scope of the present invention thereto but are intended to exemplify the present invention unless otherwise specified. The relative positions or the size of the elements illustrated in the drawings may be exaggerated for purpose of clear illustration.

In recent years, services that integrate cyberspace and real space have attracted attention. An eyeglasses image display device such as a retinal projection display has been developed and commercialized so as to play a central role in the services.

The eyeglasses image display device is roughly divided into an immersive display and a see-through display. A line-of-sight detector is embedded in the immersive eyeglasses image display, and such a device becomes a standard. By contrast, in the see-through eyeglasses image display, the installation of various sensors and cameras is minimized in order to achieve a small-sized casing, a light-weight device, or an appearance equivalent to the typical eyeglasses. Although there are commercially available devices that perform only line-of-sight detection, no display device including the line-of-sight detection device is commercially available.

At present, image processing in the eyeglasses image display device is often performed by an external terminal. In the field of augmented reality (AR), the see-through eyeglasses display device is expected to expand the market in the field of the AR in the future. Preferably, the see-through eyeglasses display device is small-sized and light weight. In addition, the see-through eyeglasses image display device can naturally access visual information without disturbing human cognitive behavior. Further, the see-through eyeglasses image display device can increase the viewing angle and the image clarity and achieve higher performance and diversity of the human-machine interface.

A technique to detect an inclination of the eyeball by using a micro electro mechanical systems (MEMS) mirror as a smaller line-of-sight detector is known. The line-of-sight detector using the MEMS mirror does not use a display device such as a digital micromirror device (DMD), which is referred to as a non-image method. In the line-of-sight detector using the MEMS mirror, the inclination of the eyeball is detected based on the light receiving signal of the reflected light from the eyeball. The non-image method does not use an image. In the non-image method, there is a technique to estimate a rotation angle of the eyeball (i.e., cycloduction). In the technique, multiple light sources emits light beams to the eyeball and the reflected light beams reflected from the eyeball are received.

However, the eyeglasses image display device accommodates: multiple sensors, driving circuits of the multiple sensors, a detection circuit, a feedback circuit, a calculation circuit and a battery in a casing equivalent to the typical eyeglasses. The line-of-sight detector also accommodates an element or a part such as a light source, a movable mirror, a light detector, electric circuits to drive and control these elements and parts, and an electric circuits to obtain information on the line-of-sight in the casing. In order to accommodate these elements and electric circuits in the casing, these element and circuit reduce the size and are mounted at higher precision.

According to the present embodiment, a line-of-sight detector that can reduced the size is provided. Further, in the present embodiment, an inclination detector mounted with higher accuracy is provided. Further, in the present embodiment, for example, the line-of sight detector is expected to be mounted on the see-through eyeglasses image display device, and the line-of-sight detector of the non-image method is provided.

Hereinafter, a description will be given of the line-of-sight detector that is mounted on a holder (an eyeglasses support) and detects the inclination angle of the eyeballs of a user wearing the holder (the eyeglasses support) as the line-of-sight direction will be described. The human eye is an example of a three-dimensional object. A line-of-sight detector detects information on the inclination of an eyeball inclined in a direction in which a human eyes are directed as information indicating the direction of the human eyes (information on the line-of-sight direction). The inclination information of the eyeball includes information relevant to an inclination angle other than the inclination angle of the eyeball in addition to information indicating a direct inclination angle.

In an inclination detector includes: a first support including: a first surface; and a second surface opposite to the first surface; a light source on the first surface, the light source to emit a light beam; a light guide facing the light source, in which the light guide guides the light beam emitted from the light source; a light exit portion to emit the light beam through the first support, in which the light beam guided from the light source on the first surface toward the second surface through the light guide; a first light receiver on the second surface of the first support, in which the first light receiver receives the light beam emitted from the light exit portion and reflected from a three-dimensional object and outputs a first reception signal; and circuitry to detect an inclination of the three-dimensional object based on the first reception signal output from the first light receiver.

In the following description, the eyeball of the right eye of a human is exemplified, but the same applies to the eyeball of the left eye. In addition, two line-of-sight detectors can be applied to the eyeballs of both eyes respectively. The three dimensional object is not limited to an eyeball, and the present embodiment can be applied as long as it is a three-dimensional object having curvature.

### EMBODIMENT

### Configuration Example of Line-of-Sight Detector

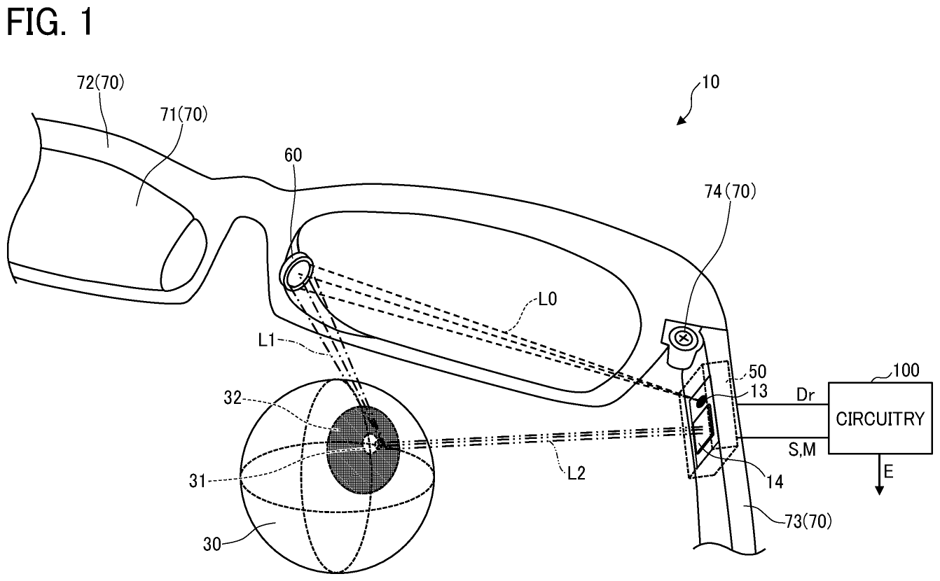

FIG. 1 is a diagram of an overall configuration of the line-of-sight detector 10. The line-of-sight detector 10 includes an optical unit 50, a reflective light condenser 60, a holder 70 (an eyeglasses support), and circuitry 100 (processing circuitry, circuits). The holder 70 (the eyeglasses support) includes a lens 71, a rim 72 (eyeglasses frame), a temple 73, and a hinge 74. The rim 72 holds the lens 71. The hinge 74 connects the rims 72 and the temple 73 so that the rim 72 and the temple 73 can move. The temple 73 holds the optical unit 50 by housing the optical unit 50 inside the temple 73. The holder 70 (the eyeglasses support) is an example of a holding member to hold the first support included in the optical unit 50. In a line-of-sight detector including the inclination detector according to the embodiments, the three-dimensional object includes an eyeball, and the line-of-sight detector detects a line-of-sight of the eyeball.

The holder 70 (the eyeglasses support) can be mounted on a user head. When the holder 70 (the eyeglasses support) is attached on the user's head (i.e., human head), the optical unit 50 is disposed at a position (in front of the eye) close to the three-dimensional object 30 (the eyeball) of the user. The optical unit 50 includes a light source, a light exit portion 13, and a first light receiver 14. The light source emits the laser light beam L0 in response to the driving signal Dr from the circuitry 100, and the optical unit emits the laser light beam L0 to the reflective light condenser 60 through the light exit portion 13.

The reflective light condenser 60 is provided on the holder 70 (the eyeglasses support). The reflective light condenser 60 reflects the laser light beam L0 from the light exit portion 13 toward the three-dimensional object 30 (the eyeball) which condensing the laser light beam L0. The reflective light condenser 60 is, for example, a concave mirror. The reflective light condenser 60 is provided on the rim 72 so that when the holder 70 (the eyeglasses support) is worn on the face of the user, the reflective light condenser 60 is positioned near the nose of the user wearing the holder 70 (the eyeglasses support). The converging light beam L1 condensing by the reflective light condenser 60 hits a portion closer to the pupil 31 of the three-dimensional object 30 (the eyeball).

The incident angle of the converging light beam L1 on the three-dimensional object 30 (the eyeball) is adjusted so as to be incident on the center of the pupil 31 of the three-dimensional object 30 (the eyeball) at a predetermined angle at the time of emmetropia. The surface of the pupil 31 (i.e., the surface of the cornea 32) of the three-dimensional object 30 (the eyeball) is a transparent body containing moisture and typically has a reflectance of about 2 to 4%. The converging light beam L1 entering in the vicinity of the pupil 31 of the three-dimensional object 30 (the eyeball) is reflected on the surface of the pupil 31 of the three-dimensional object 30 (the eyeball). The reflected light beam L2 from the pupil 31 enters the first light receiver 14 of the optical unit 50. The second light receiver 12 outputs the reception signal S (light reception signal) of the reflected light beam L2 to the circuitry 100.

The circuitry 100 estimates the rotation angle of the three-dimensional object 30 (the eyeball) based on the reception signal S. The circuitry 100 outputs the line-of-sight information E of the three-dimensional object 30 (the eyeball) corresponding to the rotation angle. The circuitry 100 controls the light amount of the laser light beam L0 emitted from the light source by outputting a drive signal Dr based on the light amount monitoring signal M from the optical unit 50.

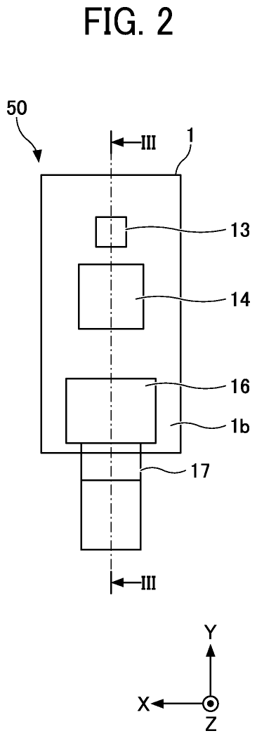

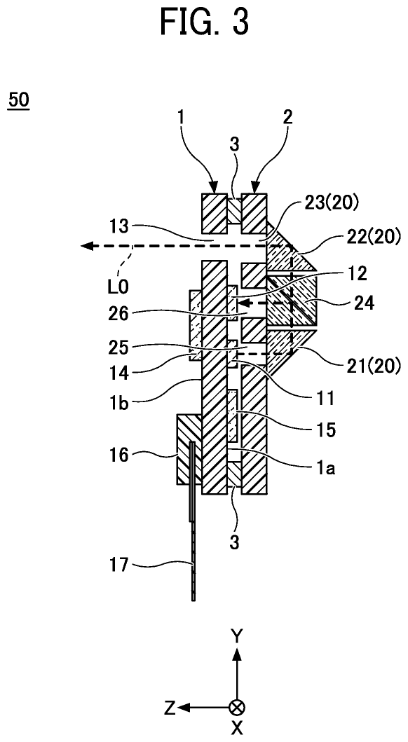

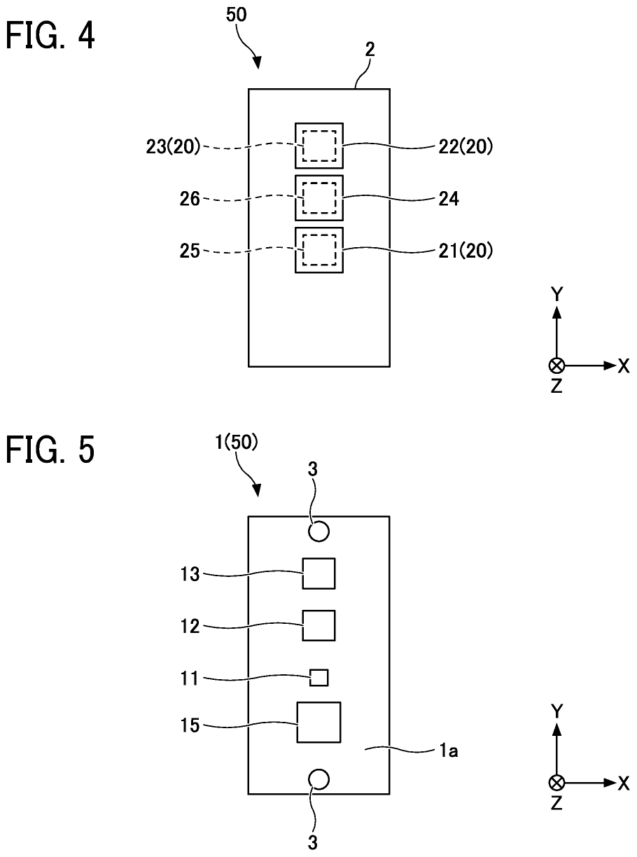

FIGS. 2 to 4 are diagrams of the configuration of the optical unit 50 of the line-of-sight detector 10. FIG. 2 is a bottom view of the optical unit. FIG. 2 is a diagram as viewed from the three-dimensional object 30 (the eyeball) to the optical unit 50 in FIG. 1. FIG. 3 is a cross-sectional view of the optical unit 50 taken along the line III-III in FIG. 2. FIG. 4 is a top view of the optical unit 50. FIG. 5 is a top view of a first support 1 in a state where a second support 2 is removed from the optical unit 50.

As illustrated in FIG. 3, the optical unit 50 includes the first support 1 (the first substrate), the second support 2, a spacer 3, a light source 11, a light guide 20, a light exit portion 13, and the first light receiver 14. The light guide 20 includes a first light guide 21 (a first prism), a second light guide 22 (a second prism 22), and a first through hole 23.

The first support 1 is an example of a support including a first surface 1a and a second surface 1b. The first support 1 is a mounting substrate on which the light source 11 and the first light receiver 14 are mounted. The second surface 1b is a surface opposite to the first surface 1a of the first support 1. The light source 11, a second light receiver 12, and an amplifier 15 are mounted on the first surface 1a of the first support 1. A first light receiver 14 and a connector 16 are mounted on the second surface 1b of the first support 1. The connector 16 is connected to the flexible substrate 17. The light exit portion 13 is a through hole penetrating the first support 1.

In the inclination detector according to the embodiments, the first support includes a circuit board mounting the light source on the first surface and mounting the first light receiver on the second surface.

The second support 2 is an example of a second support that supports at least the light guide 20. The second support 2 faces the first support 1 and is provided so as to overlap the first support 1 as viewed from the normal direction of the first support 1. Hereinafter, viewing from the normal direction of the first support 1 is referred to as a plan view. The spacer 3 is disposed between the first support 1 and the second support 2. The second support 2 is fixed to the first support 1 by an adhesive member via a spacer 3.

The inclination detector the embodiments, further includes: a second support having a third surface and a fourth surface opposite to the third surface, the light guide on the third surface; and a spacer between the first surface of the first support and the fourth surface of the second support.

A first light guide 21 (a first prism), a second light guide 22 (a second prism), and a light separator 24 (a beam splitter) are mounted on a surface of the second support 2 opposite to the surface facing the first support 1. A first through hole 23, a second through hole 25, and a third through hole 26 are formed in the second support 2. The first through hole 23 overlaps with the through hole of the light exit portion 13 in a plan view. The second through hole 25 includes the light source 11 inside in a plan view. In other words, the light source 11 is viewed through the second through hole 25 in a plan view. The third through hole 26 includes the second light receiver 12 inside in a plan view. In other words, the second light receiver 12 is viewed through the third through hole 26 in a plan view.

The light source 11 emits a laser light beam L0. The first prism 21 reflects the laser light beam L0 emitted from the light source 11 toward the light separator 24 (the beam splitter). The light separator 24 (the beam splitter) reflects a portion of the laser light beam L0 from the first light guide 21 (the first prism) toward the second light receiver 12 and transmits the remaining of the laser light beam L0. The second light guide 22 (the second prism) reflects the laser light beam L0 transmitted through the light separator 24 (the beam splitter) toward the first through hole 23. The laser beam L0 reflected by the second light guide 22 (the second prism) passes through the first through hole 23 and the light exit portion 13, and propagates toward the reflective light condenser 60.

The first light receiver 14 outputs a reception signal S of the reflected light beam L2 reflected by the three-dimensional object 30 (the eyeball). The amplifier 15 electrically amplifies the reception signal S. The connector 16 inputs the reception signal S amplified by the amplifier 15. The optical unit 50 outputs the reception signal S input through the connector 16 to the circuitry 100 through the flexible substrate 17.

The light source 11 is a vertical cavity surface emitting laser (VCSEL) array in which VCSEL elements are two-dimensionally arrayed. The light source 11 can be driven individually for each VCSEL array. Herein, the minimum unit of an individually driven VCSEL array is referred to as a light emitting portion. Each light emitting portion emits a laser beam having directivity and a finite spread angle. The VCSEL array is an example of the light emitting portion.

The wavelength of the laser light emitted from the light source 11 preferably includes a wavelength of near-infrared light, which is invisible light, so that the visual recognition of a “user” whose line-of-sight is detected is not prevented. However, the wavelength is not limited to the near-infrared light, and the wavelength of the laser light emitted from the light source 11 may be visible light.

The light source 11 is not limited to the VCSEL array, and may be a laser diode (LD), a light emitting diode (LED), or a laser light other than the LD. Since the VCSEL array can be easily integrated in a two-dimensional plane, it is preferable that it can be mounted in a smaller size in a wearable device.

The light guide 20 guides the laser light beam L0 emitted from the light source 11. The light guide 20 may have any configuration as long as it can guide the light emitted from the light source 11. For example, the light guide 20 may include at least one of a lens, a diffraction grating, or an optical fiber.

The light exit portion 13 emits the laser light beam L0 guided from the first surface 1a through the light guide 20 to the second surface 1b. The light exit portion 13 may have any configuration as long as it can emit the light guided through the light guide 20. For example, the light exit portion 13 may include a notch or a slit through the first support 1. In addition, a portion of a lens, a diffraction grating, an optical fiber, a light transmission member, or a neutral density filter included in the light guide 20 from which light beam is emitted may be used as the light exit portion 13. The shape of the light exit portion 13 may be an ellipse, a rectangle, or a polygon in addition to the circular shape.

The light separator 24 (the beam splitter) is an example of a light splitting element that splits the laser light beam L0 emitted from the light source 11 into two or more light beams. The light separator 24 (the beam splitter) is not limited to a cube and may be a plate.

The second light receiver 12 outputs a light amount monitoring signal M corresponding to the light amount received by the second light receiver 12 among the reflected light and the transmitted light of the laser light beam L0 split by the light separator 24 (the beam splitter). As the second light receiver 12, a light receiving element such as a photodiode or an image element such as a CCD or a CMOS can be used. The second light receiver 12 is disposed on the first surface 1a of the first support 1. The second light receiver 12 outputs the light amount the monitoring signal M to the circuitry 100.

The inclination detector according to the embodiments, further includes a light separator adjacent to the light guide, the light separator to separate the light beam emitted from the light source into two or more light beams; and a second light receiver on the first surface, the second light receiver to receive at least one light beam among the two or more light beams and outputs a second reception signal based on the at least one light beam.

In the inclination detector according to the embodiments, the light guide includes a first light guide (a first prism 21) and a second light guide (a second prism 22) on the third surface of the second support, the light separator is on the third surface of the second support and is between the first light guide and the second light guide, each of the first support and the second support has a through hole as the light exit portion, the light beam passed through the first light guide, the light separator, and the second light guide passes through the through hole of each of the first support and the second support.

The first light receiver 14 is provided on the second surface 1b. The laser light beam L0 emitted from the light exit portion 13 hits the three-dimensional object 30 (the eyeball) and is reflected by the three-dimensional object 30 (the eyeball) as the reflected light beam L2. The first light receiver 14 receives the reflected light beam L2 reflected from the three-dimensional object 30 (the eyeball) and outputs a reception signal S. In the present embodiment, the first light receiver 14 is a position sensitive detector (PSD) to output both a signal indicating the light intensity of the received light and a signal indicating the position of the reflected light beam L2 incident on the first light receiver 14. However, the first light receiver 14 may be a single-pixel photo detector (PD) that outputs a signal indicating the light intensity of the received light, or may be an imaging device such as a charge coupled device (CCD) or a complementary metal oxide semiconductor (CMOS). The PSD suitably detects the reflected light beam L2 reflected from the three-dimensional object 30 (the eyeball) with higher accuracy and a direction of the line-of-sight based on the position on which the reflected light is incident and the position of the light emitting portion in the light source 11.

The arrangement of the reflective light condenser 60 and the first light receiver 14 is not limited to those in FIGS. 1 and 3. The arrangement of the reflective light condenser 60 and the first light receiver 14 may be such that only one of the laser light beams L0 emitted from the multiple light emitting portions of the light source 11 is finally incident on the first light receiver 14 in accordance with the inclination of the three-dimensional object 30 (the eyeball). For example, a deflection optical element may be provided between the three-dimensional object 30 (the eyeball) and the first light receiver 14.

The circuitry 100 is an example of an output unit that outputs line-of-sight information on the three-dimensional object 30 (the eyeball) based on the reception signal S output from the first light receiver 14. The circuitry 100 sequentially emits each light emitting portion of the light source 11 to emit light by outputting the drive signal Dr. The circuitry 100 executes processing to estimate the line-of-sight direction based on the reception signal S input from the first light receiver 14. The circuitry 100 is disposed in, for example, the temple 73, but is not particularly limited thereto.

Which one of the laser beams L0 emitted from the light emitting portions included in the light source 11, the reflected light beam L2 incident on the first light receiver 14 is derived from varies depending on the line-of-sight direction. Thus, the circuitry 100 estimates the line-of-sight direction based on the reception signal S of the first light receiver 14 and the position of the light emitting portion in the light source 11. The circuitry 100 estimates the line-of-sight direction using the position of the light-emitting portion in the light source 11, the position of the reflected light beam L2 incident on the first light receiver 14, and a predetermined eyeball model. The circuitry 100 outputs the line-of-sight information E based on the estimated line-of-sight direction.

The light source 11 includes multiple light emitting portions and performs time modulation at higher speed. The line-of-sight detector 10 modulates the laser light beam L0 emitted by the light source 11 with time modulation according to, for example, an encoded pattern having orthogonality, for example. The line-of-sight detector 10 extracts a component having an encoded pattern suitable for the reflected light beam L2 incident on the first light receiver 14 from the reception signal S of the first light receiver 14. Thus, the line-of-sight detector 10 can remove the influence of the light from the external environment without modulation and increase the signal-to-noise ratio (S/N) of the output signal. A line-of-sight detector 10 can easily detect the line-of-sight direction in a bright environment and can reduce the amount of the conversing light beam L1 irradiated the three-dimensional object 30 (the eyeball). When the amount of the conversing light beam L1 is decreased, the safety to the three-dimensional object 30 (the eyeball) is increased.

The line-of-sight detector 10 sequentially emits each light emitting portion in a light source 11 (i.e., sequential light emission). The sequential light emission is advantageous in reducing the light intensity of the conversing light beam L1 that irradiates the eyeball as compared with the case where each light emitting portion emits light in parallel. When all of the multiple light emitting portions emit in parallel, a light source driver such as light source modulation units corresponding to the number of the light emitting portions are prepared. Since the line-of-sight detector 10 emits light one by one, the number of the light source driver such as light source modulation units may not match the number of the light emitting portions. Thus, in the line-of-sight detector 10, the light source driver can be mounted in a small size and a light weight.

In the present embodiment, the optical unit 50 and the circuitry 100 are accommodated in the temple 73, but the present invention is not limited thereto, and a head-mounted display, or a headgear holding structure may be used.

### Hardware Configuration Example of Circuitry

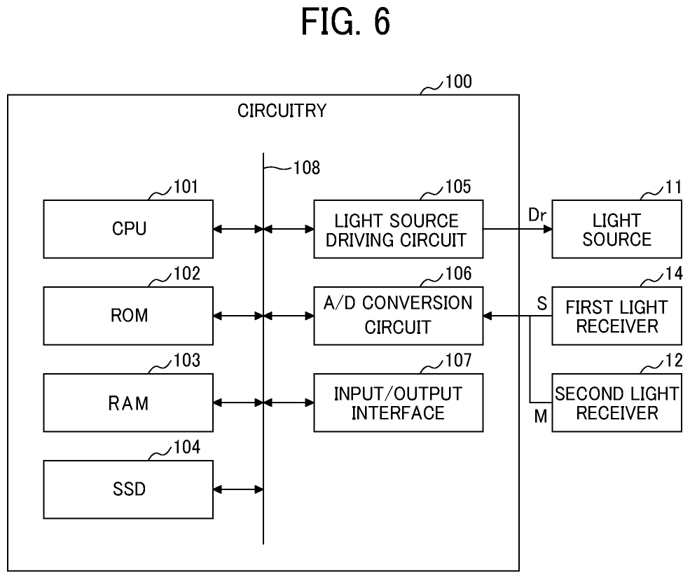

FIG. 6 is a block diagram of the hardware configuration of the circuitry 100. The circuitry 100 includes a central processing unit (CPU) 101, a read only memory (ROM) 102, a random access memory (RAM) 103, and a solid state drive (SSD) 104. The circuitry 100 includes a light source driving circuit 105, an analog digital (A/D) conversion circuit 106, and an input/output (I/O) interface (I/F) 107. These devices are connected to each other through a system bus 108 so that they can communicate with each other.

The CPU 101 reads programs and data from a storage device such as the ROM 102 or the SSD 104 in the RAM 103. The CPU 101 executes the read program to control the entire circuitry 100 and enables functions described later to work. At least some of the functions of the CPU 101 may be implemented by an electronic circuit such as an application specific integrated circuit (ASIC) or a field-programmable gate array (FPGA).

The ROM 102 is a non-volatile semiconductor memory (storage device) to store programs and data even when power is turned off. The ROM 102 stores a basic input/output system (BIOS) to be executed in response to activation of the circuitry 100, programs such as OS settings and network settings, and data. The RAM 103 is a volatile semiconductor memory (storage device) for temporarily storing programs and data.

The SSD 104 is a non-volatile memory to store a program to execute processing by the circuitry 100 and various data. The SSD may be a hard disk drive (HDD).

The light source driving circuit 105 is an electric circuit that is electrically connected to the light source 11 and outputs a drive voltage to the light source 11 in accordance with the drive signal Dr. The light source driving circuit 105 sequentially enables multiple light emitting portions of the light source 11 to emit a light beam.

A rectangular wave, a sinusoidal wave or a voltage waveform having a predetermined waveform can be used as the drive voltage, and the light source driving circuit 105 can change the period (frequency) of these voltage waveforms to modulate the period of the drive voltage.

The A/D conversion circuit 106 is electrically connected to each of the first light receiver 14 and the second light receiver 12. The A/D conversion circuit 106 outputs digital voltage data obtained by A/D conversion of the reception signal S that is an analog voltage signal output from the first light receiver 14. The A/D conversion circuit 106 outputs digital voltage data obtained by A/D conversion of the light amount monitoring signal M that is an analog voltage signal output from the second light receiver 12.

The input/output I/F 107 is an interface to connect to an external device such as a personal computer (PC) or a video device.

### Functional Configuration Example of Circuitry

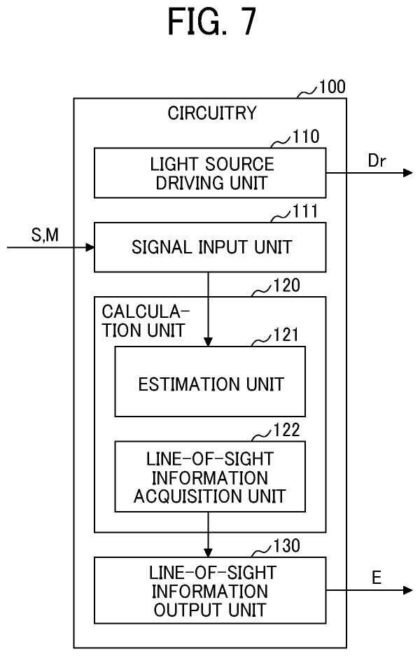

FIG. 7 is a block diagram of the functional configuration of the circuitry 100. As illustrated in FIG. 7, the circuitry 100 includes a light emission driving unit 110, a signal input unit 111, a calculation unit 120, and a line-of-sight information output unit 130.

The light emission driving unit 110 outputs the driving signal Dr to the light source 11 to enable the multiple light emitting portions of the light source 11 to emit the laser light beam L0. The light emission driving unit 110 controls the light amount of the laser light beam L0 emitted from the light source 11 based on the light amount monitoring signal M from the signal input unit 111. The light source driving circuit 105 enables the light emission driving unit 110 to work.

The signal input unit 111 outputs a digital voltage signal obtained by A/D conversion of the reception signal S input from the first light receiver 14 to the estimation unit 121 (the eye rotation angle estimation unit) included in the calculation unit 120. The signal input unit 111 outputs a digital voltage signal obtained by A/D conversion of the light amount monitoring signal M input from the second light receiver 12 to the light emission driving unit 110. The A/D conversion circuit 106 enables the signal input unit 111 to work.

The calculation unit 120 includes an estimation unit 121 (an eye rotation angle estimation unit) and a line-of-sight information acquisition unit 122. On the basis of the reception signal S input through the signal input unit 111, a calculation process to acquire the pupil position of the three-dimensional object 30 (the eyeball) is executed. The CPU 101 executes a program stored in the ROM 102 to enable the calculation unit 120 to work.

The estimation unit 121 (the eye rotation angle estimation unit) estimates the rotation angle of the three-dimensional object 30 (the eyeball) based on the reception signal S from the signal input unit 111. The estimation unit 121 (the eye rotation angle estimation unit) outputs the estimated rotation angle data to the line-of-sight information acquisition unit 122. The line-of-sight information acquisition unit 122 executes a process of acquiring position information of the pupil 31 based on the rotation angle information of the three-dimensional object 30 (the eyeball) from the estimation unit 121 (the eye rotation angle estimation unit). The line-of-sight information acquisition unit 122 executes processing to acquire the line-of-sight information E from the position of the pupil 31. The line-of-sight information acquisition unit 122 outputs the line-of-sight information E to an external device through the line-of-sight information output unit 130. The line-of-sight information output unit 130 is implemented by the input/output I/F 107.

In the line-of-sight detector 10, an incident angle at which the conversing light beam L1 emitted from a light source 11 to a three-dimensional object 30 (the eyeball) strikes the three-dimensional object 30 (the eyeball), and a formula to calculate the rotation angle of the three-dimensional object 30 (the eyeball) are predetermined. The formula to calculate the rotation angle of the three-dimensional object 30 (the eyeball) includes a first order function or a second order function. However, the form of the equation is not particularly limited as long as the rotation angle can be determined based on the incident angle of the conversing light beam L1 and the position of the first light receiver 14 on the light receiving surface. In the present embodiment, a calculation formula using a second order function is employed as a simple approximate formula.

The surface shape model of the three-dimensional object 30 (the eyeball) can be used to determine the angle at which the conversing light beam L1 enters the three-dimensional object 30 (the eyeball). For example, a simplified model eye, which has been known for a long time as a surface shape model of a typical eyeball, can be used. For example, the model of a typical eyeball is described in NPL 1.

The incident angle of the conversing light beam L1 to the three-dimensional object 30 (the eyeball) is determined in advance by ray tracing calculation so that the incident position of the reflected light beam L2 to the first light receiver 14 is at the center of the light receiving surface. The incident position of the reflected light beam L2 on the light receiving surface of the first light receiver 14 is determined by theoretical analysis based on the incident angle of the conversing light beam L1 on the three-dimensional object 30 (the eyeball), the reflection position of the conversing light beam L1 on the three-dimensional object 30 (the eyeball), and the inclination of the contact surface of the three-dimensional object 30 (the eyeball). From the solution of the theoretical analysis, an inverse operation expression (i.e., approximate expression) to estimate the rotation angle of the three-dimensional object 30 (the eyeball) by polynomial approximation is determined.

An inverse operation equation to estimate the incident angle of the conversing light beam L1 to the three-dimensional object 30 (the eyeball) and the rotation angle of the three-dimensional object 30 (the eyeball) is stored in a memory such as the ROM 102 or the SSD 104 of the circuitry 100. The inverse calculation formula is referred to in changing the light emitting portion by the light emission driving unit 110 and in acquiring the line-of-sight information E by the calculation unit 120.

### First Embodiment

### Configuration Example of Light Source

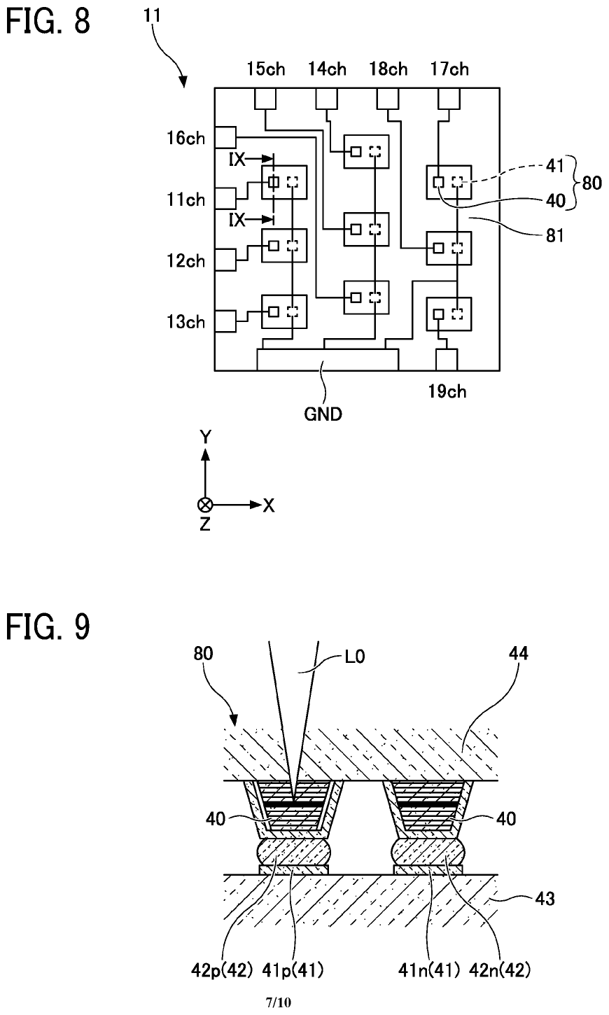

The configuration of the light source 11 will be described with reference to FIGS. 8 and 9. FIG. 8 is a configuration of the light source 11 according to the first embodiment. FIG. 9 is a cross-sectional view taken along the line IX-IX in FIG. 8.

As illustrated in FIG. 8, the light source 11 includes nine VCSEL arrays 80 of the 11 channel (11ch) to 19 channel (19ch) each including a light emitting portion 40. The light emitting portion 40 of the VCSEL array 80 corresponds to an individual light emitting portion. The term “individual” refers to a state of complete disconnection between the light emitting portion. In other words, multiple light emitting portions are separated from each other. An air layer 81 is provided between the multiple light emitting portions 40. In other words, the air layer 81 is a space or an interval of air. The air layer 81 is a layer in which air exists between the VCSEL arrays 80 including the light emitting portions 40.

In the inclination detector according to the embodiments, the light source includes multiple light emitting portions.

In the inclination detector according to the embodiments, the light source includes multiple light emitting portions each of which are spaced from each other.

In the present embodiment, the light source 11 is not a VCSEL array including multiple light emitting portions 40 in one chip. The light source 11 is a VCSEL array 80, which is an individual chip for each light emitting portion 40, is produced in the wafer surface. The VCSEL array 80 is selectively picked up using a patterned adhesive and transferred onto the donor substrate. Such a manufacturing process of the light source 11 is referred to as a microtransfer process.

When multiple VCSEL arrays 80 are used for the light source 11, an effect of reducing the number of optical members can be obtained by optimizing the interval between the VCSEL arrays 80. By contrast, it is not preferable to manufacture a configuration in which the VCSEL array 80 having the non-periodic structure is arranged at relatively sparse intervals by a semiconductor manufacturing process. Since the number of VCSEL arrays 80 taken out per wafer is small, the mass production effect cannot be obtained.

Since the multiple VCSEL arrays 80 are manufactured by the microtransfer process, the light source 11 can dispose the multiple VCSEL arrays 80 without waste. For space saving and miniaturization, it is preferable that the VCSEL array 80 in the light source 11 includes an electrode configuration corresponding to flip-chip mounting. FIG. 8 is a diagram of a configuration in which one electrode is common and connected to GND.

As illustrated in FIG. 9, the VCSEL array 80 includes a light emitting portion 40, an electrode 41, a conductor 42, a first semiconductor substrate 43 (a first substrate), and a second semiconductor substrate 44 (a second substrate). The electrode 41 includes an anode terminal 41p and a cathode terminal 41n. The conductor 42 includes a conductive member 42p and a conductive member 42n. The conductive member 42p connects the light emitting portion 40 and the anode terminal 41p. The conductive member 42n connects the light emitting portion 40 and the cathode terminal 41n.

The VCSEL array 80 has a shape obtained by processing a multilayer film into a mesa. The VCSEL array 80 can be used as an n-type electrode in FIG. 9, for example, by covering the mesa structure in the adjacent light emitting portion 40 with the conductor 42 made of metal. Since the conductor 42 is melted and compressed as a bump, the electrode 41 and the light emitting portion 40 are electrically connected to each other, and current can be injected into the VCSEL array 80.

When the number of the VCSEL arrays 80 is larger, it is preferable to provide the common electrode in different layers by using a multilayer substrate having a through hole, because wiring lines cross each other. Thus, it is preferable that the cathode terminal 41n and the anode terminal 41p are electrically connected to different layers of the double-sided substrate or the multilayer substrate.

In the inclination detector according to the embodiments, the light source includes: a first light-source substrate (a first semiconductor substrate); a second light-source substrate (a second semiconductor substrate); the multiple light emitting portions between the first light-source substrate and the second light-source substrate; a cathode terminal on the first light-source substrate; and an anode terminal on the first light-source substrate. The cathode terminal and one of the multiple light emitting portions are between the first light-source substrate and the second light-source substrate to electrically connect the first light-source substrate and the second light-source substrate, and the cathode terminal and another of the multiple light emitting portions are between the first light-source substrate and the second light0source substrate to electrically connect the first light-source substrate and the second light-source substrate.

### Operation and Effect of Line-of-Sight Detector

The operation and effect of the line-of-sight detector 10 will be described. For example, PTL 1 discloses an optical scanning module in which multiple reflective surfaces, edge light-emitting laser diodes, a monitor photodiode, and a collimator lens are laminated on a flat substrate. The optical scanning module distributes a laser light beam so as to surround a scanning mirror. The optical scanning module irradiates a scanning mirror with the laser light beam and transmits the laser light beam through a transparent glass cover.

However, in the configuration described in PTL 1, the scanning mirror is three-dimensionally arranged so as to obtain a preferable exit angle. Further, in order to obtain a movable space for the scanning mirror, a void space is provided. Further, since an edge-emitting laser diode is used, a height is adjusted by a submount. The alignment with the collimator lens is also not easy. Since an edge-emitting laser diode distorts a cross-sectional profile of a light beam, an optical element for beam shaping is provided. Accordingly, when the configuration described in PLT1 is used, the line-of-sight detector 10 may increase in size.

The line-of-sight detector 10 (inclination detection device) according to the present embodiment comprises a first support 1 (first support), a light source 11, a light guide 20, a light exit portion 13, a first light receiver 14, and circuitry 100 (output section). The light source 11 is provided on the first surface 1a of the first support 1. The first light receiver 14 is provided on the second surface 1b of the first support 1. According to the configuration, on the first support 1, the light source 11, an electronic circuit for driving the light source 11, an electronic circuit for processing a signal from the first light receiver 14 can be arranged on the surface opposite to the surface on which the first light receiver 14 is provided. As a result, the line-of-sight detector 10 can be reduced in size.

Further, since the line-of-sight detector 10 can apply a configuration that does not include any movable part. Thus, a space for a range of motion may be omitted. Further, the line-of-sight detector 10 may omit a driving circuit or a power supply to drive and control the movable mechanism. Thus, the line-of-sight detector 10 can be reduced in size.

The first light receiver 14 preferably has a larger light receiving area, because the larger light receiving area has an advantage described below. The laser light beam L0 is emitted to the three-dimensional object 30 (the eyeball). The reflected light beam L2 is incident on the light receiving surface of the first light receiver 14. The reflected light beam L2 strikes a position on the light receiving surface of the first light receiver 14 corresponding to the inclination of the three-dimensional object 30 (the eyeball). The reflected light beam L2 spreads in accordance with the surface shape of the three-dimensional object 30 (the eyeball) and strikes the light receiving surface of the first light receiver 14. Thus, if the light receiving area of the light receiving surface of the first light receiver 14 is larger, it is advantageous to detect the position of the reflected light beam L2 and the amount of the reflected light beam L2. As described above, it is preferable that the light receiving surface of the first light receiver 14 has a larger light receiving area.

Further, in the present embodiment, the first support 1 is a mounting substrate on which the light source 11 and the first light receiver 14 are mounted. According to the configuration, the light source 11 and the first light receiver 14 are electrically connected and can be integrated and mounted on the first support 1, so that the optical unit 50 is more likely to handle.

Herein, the optical unit 50 includes the first support 1 and the second support 2 in the present embodiment. However, the optical unit 50 may not include the second support 2. When the optical unit 50 does not include the second support 2, the light guide 20 and the light separator 24 (the beam splitter) are disposed on the first support 1.

In the present embodiment, the light source 11 includes multiple light emitting portions 40. By switching the light emission of each of the multiple light emitting portions 40 and switching the incidence angle of the laser light beam L0 on the three-dimensional object 30 (the eyeball), tracking the movement of the three-dimensional object 30 (the eyeball) is achieved.

In the present embodiment, an air layer 81 is provided between the multiple light emitting portions 40. According to the configuration, since the multiple light emitting portions 40 are suitably arranged, an optical element to control the incident position of the reflected light beam L2 to the first light receiver 14 and, controlling the shape of the laser beam of the reflected light beam L2 is omitted. As a result, the number of optical components is substantially decreased. Further, an optical element can be mounted by mounting accuracy of an electronic element or mounting accuracy using an electronic element mounting device. As a result, the line-of-sight detector 10 including a small number of optical elements, which is easily mounted, can be provided.

Preferably, a light source 11 including multiple light emitting portions 40 that emit linearly polarized light having a proper polarization azimuth is used. Since the polarization of the laser incident on the three-dimensional object 30 (the eyeball) is uniquely determined, the amount of the light entering the three-dimensional object 30 (the eyeball) is decreased without providing a special polarizing element.

In addition, in the present embodiment, the optical unit 50 includes a light separator 24 (a beam splitter) (light splitting member) and a second light receiver 12. The second light receiver 12 is disposed on the first surface 1a of the first support 1. According to the configuration, a portion that can omit electrical connection such as the light guide 20 and the light separator 24 (the beam splitter), can be integrated into the second support 2, so that the optical unit 50 can be handled easily.

In the present embodiment, the light emitting portion 40 includes a cathode terminal 41n and an anode terminal 41p. The cathode terminal 41n and the anode terminal 41p may be electrically connected to different layers of the double-sided substrate or the multilayer substrate. According to the configuration, the wiring space of the light source 11 is reduced, the gap area is effectively utilized, and the line-of-sight detector 10 is reduced in size.

Further, in the present embodiment, the optical unit 50 includes a second support 2 supporting at least the light guide 20 and a spacer 3 disposed between the first support 1 and the second support 2. The light guide 20 and the light separator 24 (the beam splitter), which are optical elements having a flat incident/exit surface allow a higher mounting accuracy. Thus, the mounting accuracy is sufficient by laminating the first support 1 and the second support 2 using the spacer 3. As a result, the line-of-sight detector including the small number of optical elements, which is easily mounted, is provided.

### Second Embodiment

A light source of the line-of-sight detector according to the second embodiment will be described. The same components are denoted by the same reference numerals, and redundant description will be appropriately omitted. This point is also applied to other embodiments described below.

### Configuration Example of Light Source

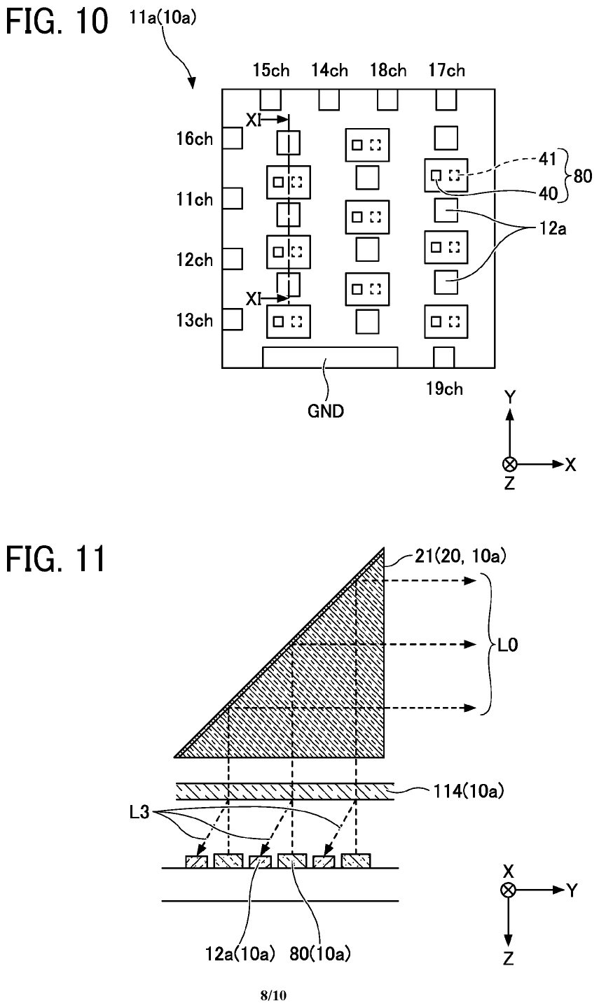

With reference to FIGS. 10 and 11, the configuration of the light source 11a of the line-of-sight detector 10a according to the second embodiment will be described. FIG. 10 is a diagram of a configuration of a light source 11a according to the second embodiment. FIG. 11 is a cross-sectional view taken along the line X-X in FIG. 10.

As illustrated in FIG. 10, the light source 11a includes multiple VCSEL arrays 80 and multiple second light receiver 12a. Each of the multiple VCSEL arrays 80 has a single light emitting portion 40. The multiple second light receivers 12a are provided at positions adjacent to each of the multiple VCSEL arrays 80. The multiple second light receives 12a are paired with the multiple VCSEL arrays 80. The second light receiver 12a is, for example, a photodiode, but it is not limited thereto as long as it can output an electric signal corresponding to the light intensity.

The second light receiver 12a is disposed between the multiple light emitting portions 40. Since the arrangement of the VCSEL array 80 is sparse, the same number of the second light receiver 12a as the VCSEL array 80 can be disposed adjacent to the VCSEL array 80. The line-of-sight detector 10 individually detects the light amount of the laser light beam L0 emitted from each of the VCSEL array 80 by the second light receiver 12a.

In order to detect the laser light beam L0 emitted from the VCSEL array 80 by the second light receiver 12a, a portion of the laser light beam L0 is deflected in the direction in which the second light receiver 12a is disposed

In the present embodiment, as illustrated in FIG. 11, the line-of-sight detector 10a includes a light deflector 114 (a polarization anisotropic element) between the first light guide 21 (the first prism) and the VCSEL array 80. The polarization anisotropic element 114 is an example of a deflecting element that deflects light from each of the multiple light emitting portions. The light deflector 114 (the polarization anisotropic element) deflects the laser light beam L0 emitted from each of the multiple VCSEL arrays 80 so as to be incident on the second light receiver 12a paired with the VCSEL array 80.

In the inclination detector according to the embodiments, further includes: a light deflector to deflect a light beam emitted from the light source. The light source includes multiple light emitting portions separated from each other; the light deflector deflects each light beam respectively emitted from the multiple light emitting portions, the second light receiver includes multiple second light receivers separated from each other. The multiple second light receivers make pairs with the multiple light emitters, in which the second light receiver and the light emitters are alternately disposed, and the light deflector deflects a light beam emitted from each of the multiple light emitting portions to the second light receiver made a pair with the light emitter.

In terms of two polarization components orthogonal to each other, the light deflector 114 (the polarization anisotropic element) transmits one polarization component and deflects the other polarization component by diffraction to cause the deflected polarization component to enter the second light receiver 12a. In terms of the right-handed polarization and the left-handed polarization, the light deflector 114 (the polarization anisotropic element) transmits either the right-handed polarization or the left handed polarization and deflects the remaining of the polarization by diffraction to cause the deflected polarization to enter the second light receiver 12a. The first order diffraction light beam L3 of the laser light beam L0 emitted from the VCSEL array 80 diffracted by the light deflector 114 (the polarization anisotropic element) enters the second light receiver 12a.

The light deflector 114 (the polarization anisotropic element) can generate polarization anisotropy depending on the alignment pattern of the liquid crystal by, for example, aligning liquid crystal molecules and polymerizing the liquid crystal molecules. In this case, it is sufficient to detect the light amount, so that the diffraction may not have a higher efficiency.

The line-of-sight detector including the light source 11a enables the line-of-sight detector 10 to detect the amount of the laser light beam L0 in the line-of-sight detector 10, and the light separator 24 (the beam splitter), a second light receiver 12, and a third through hole 26 in the line-of-sight detector 10 can be omitted. Thus, the line-of-sight detector can further reduce the size.

Effects other than the above are the same as those of the first embodiment.

### Third Embodiment

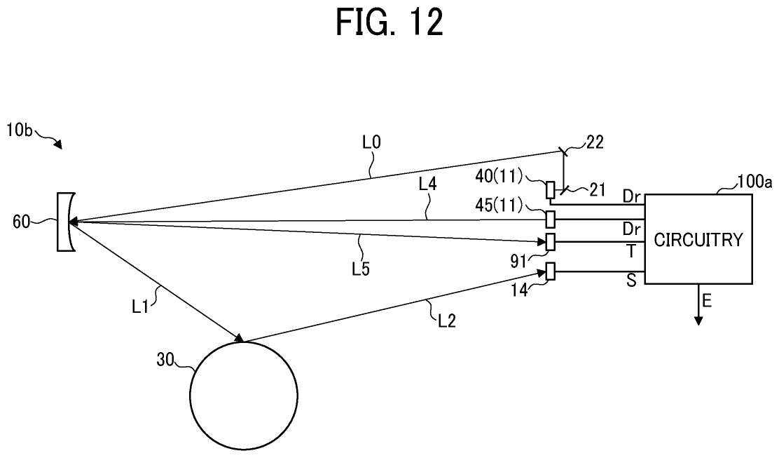

FIG. 12 is a diagram of the configuration of the line-of-sight detector 10b according to the third embodiment. The line-of-sight detector 10b includes a reflective light condenser 60 held by a holder 70 (an eyeglasses support), and a third light receiver 91 to receive light from the reflective light condenser 60.

The light source 11 includes a light emitting portion 40 as a first light emitting portion and a light emitting portion 45 as a second light emitting portion. The laser light beam L0 emitted from the light emitting portion 40 is reflected by the reflective light condenser 60 and travels while conversing, and becoming the converging light beam L1. The converging light beam L1 reflected by the eyeball becomes the reflected light beam L2. The first light receiver 14 outputs the reception signal S of the reflected light beam L2. The light emitting portion 45 emits laser light beam L2 to the reflective light condenser 60. The laser light beam L2 is reflected by the reflective light condenser 60 while conversing and becomes the converging light beam L5. The third light receiver 91 outputs the reception signal T of the converging light beam L5.

The inclination detector according to the embodiments, further includes: a holder to hold the first support; a reflective light condenser held by the holder, the reflective light condenser to reflect the light beam emitted from the light exit portion to the three-dimensional object while condensing the light beam; a third light receiver to receive the light beam reflected from the reflective light condenser; and circuitry to detect the inclination of the three-dimensional object. The light source includes: a first light emitting portion; and a second light emitting portion, the first light receiver receives the light beam emitted from the first light emitting portion and reflected from the three-dimensional object to output the first reception signal, the third light receiver receives the light beam emitted from the second light emitting portion and reflected from the reflective light condenser to output a third reception signal, and the circuitry detects the inclination of the three-dimensional object, in which a position and an angle of the reflective light condenser are compensated based on each of the first reception signal and the third reception signal.

The output of the reception signal S by the first light receiver 14 and the output of the reception signal T by the third light receiver 91 may be performed by one light receiver. At this time, since the reception signal T and the reception signal S are different in time-series patterns, each pattern of the signals can be distinguished by calculating the inner product of the respective patterns.

The circuitry 100a outputs line-of-sight information E in which the position and the angle of the reflective light condenser 60 are compensated based on the reception signal S from the first light receiver 14 and the reception signal T (light reception signal) from the third light receiver 91. For example, the circuitry 100a corrects an inverse operation formula stored in a memory such as the ROM 102 or the SSD 104 based on the reception signal T, and thereby outputting the line-of-sight information E in which the position and angle of the reflective light condenser 60 are compensated. The inverse operation formula estimates the incident angle of the conversing light beam L1 to the three-dimensional object 30 (the eyeball) and the rotation angle of the three-dimensional object 30 (the eyeball).

For example, when the user wears the holder 70 (the eyeglasses support), the position or angle of the reflective light condenser 60 may be displaced. In some cases, the line-of-sight detector may not correctly detect the line-of-sight direction of the user due to the positional displacement or the angular change of the reflective light condenser 60. In the present embodiment, the reception signal T of the conversing light beam L5 is used. The conversing light beam L5 that does not pass through the three-dimensional object 30 (the eyeball) and from the third light receiver 91 is used. Accordingly, a displacement in the position or a change in angle of the reflective light condenser 60 is detected, and the influence on the line-of-sight information can be compensated. As a result, according to the present embodiment, the line-of-sight detector 10b having a higher robustness of the line-of-sight detection is provided.

Effects other than the above are the same as those of the first embodiment.

### Fourth Embodiment

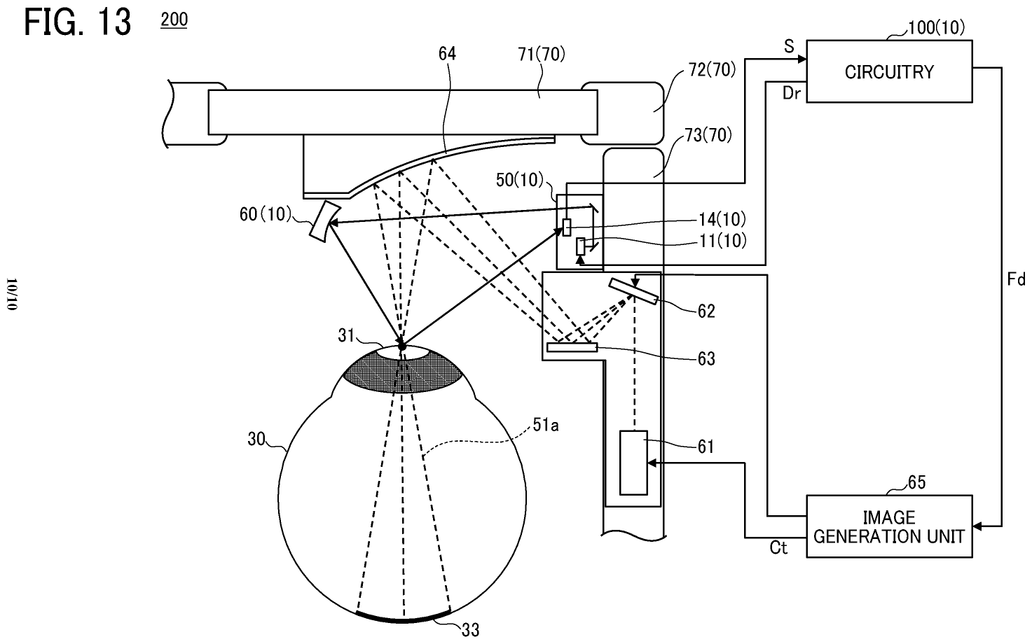

FIG. 13 is a diagram of an example of the configuration of a retinal projection display 200 according to the fourth embodiment. The retinal projection display 200 includes a red-green-blue (RGB) laser light source 61, a scanning mirror, the plane mirror 63, a half mirror 64, an image generation unit 65, and a line-of-sight detector 10. The retinal projection display 200 projects and displays an image on the retina of a user (human) wearing the retinal projection display 200. The retinal projection display 200 is an example of a head-mounted display. In a head-mounted display including the line-of-sight detector according to the embodiments. In a retinal projection display including the line-of-sight detector according to the embodiments.

The RGB laser light source 61 modulates the three colors (i.e., red, green, and blue) of light with time and emits the three colors of light beams. The scanning mirror 62 two-dimensionally scans the retina with the light beams emitted from the RGB laser light source 61. The scanning mirror 62 is, for example, a MEMS mirror. The scanning mirror 62 is not limited to the MEMS mirror, and may be a polygon mirror, a galvano mirror, or other devices having a reflecting portion for light scanning. The MEMS mirror is advantageous in reducing size and weight. The drive system of the MEMS mirror may be any of an electrostatic system, a piezoelectric system, or an electromagnetic system.

The plane mirror 63 reflects the scanning light beam by the scanning mirror 62 toward the half mirror 64. The half mirror 64 transmits a portion of the incident light beam and reflects a remaining portion of the light beam toward the three-dimensional object 30 (the eyeball). The half mirror 64 has a curved surface of a concave shape. The half mirror 64 condensing the reflected light beam at a position in the vicinity of the pupil 31 of the three-dimensional object 30 (the eyeball) and forms an image at the position of the retina 33. Thus, an image formed by the scanning light beam is projected onto the retina 33. The light beam 61a represented by a broken line in the drawing represents the light beam to form an image on the retina 33. A ratio of the reflection light beam and the transmission light beam of the half mirror 64 is typically one to one, but may have other ratios.

The line-of-sight detector 10 transmits a feedback signal Fd indicating the inclination of the three-dimensional object 30 (the eyeball), that is, the line-of-sight direction, to the image generation unit 65.

The image generation unit 65 includes functions of controlling the swing (scan or deflection) angle of the scanning mirror 62 and the light emission of the RGB laser light source 61. Further, the image generation unit 65 receives a feedback signal Fd of the line of sight direction from the line-of-sight detector 10. By outputting a control signal Ct in response to the line-of-sight information E acquired by the line-of-sight detector 10, the deflection angle of the scanning mirror 62 and the light emission of the RGB laser light source 61 are controlled, and the projection angle of an image or the image content is rewritten. Thus, an image that follows a change in the viewing direction (i.e., eye tracking) can be formed on the retina 33.

In the present embodiment, the retinal projection display 200 is an example of the head-mounted display that is a wearable terminal, but is not limited thereto. For example, the retinal projection display 200 may be mounted directly on the human head or indirectly on the human head via a member such as a fixed portion (e.g., the head-mounted display device). Alternatively, the retinal projection display may include a pair of retinal projection displays 200 for right and left eyes (i.e., a binocular retinal projection display).

In the present embodiment, the retinal projection display 200 includes the line-of-sight detector 10 as an example. In addition, the retinal projection display 200 may include the line-of-sight detector 10a or the line-of-sight detector 10c.

### Other Preferred Embodiment

Although preferred embodiments of the present invention have been described in detail above, the present invention is not limited to these embodiments, and various modifications or changes are made within the scope of the present invention described in the claims below.

For example, in the embodiments described above, the device to detect the inclination of the three-dimensional object 30 (the eyeball) is applied to the optical device as an example, but is not limited thereto. For example, an optical device may be mounted on a robot hand, and the inclination of the robot hand as an object may be detected.

The present embodiment can also be applied to an eye test device having a function of detecting the inclination of the eyeball and the position of the pupil (cornea). Herein, “the eye test device” refers to a device to perform various examinations such as visual acuity test, a refraction test, an intraocular pressure test (tonometry), and axial length test. An eye test device, which can text the eye without contact with an eyeball, includes a support section to support the face of the subject, an optometric window, a display to display the direction of the eyeball (direction of the line of sight) of the subject at the time of optometry, a controller, and a measurement section. In order to increase the measurement accuracy of the measurement unit, the subject stares at one point without moving the eyeball (line-of-sight), and the subject fixes the face to the support and gazes at an object displayed on the display object from the optometric window. At this time, when the inclination position of the eyeball is detected, the inclination detector of the eyeball according to the present embodiment is used. The inclination detector of the eyeball is arranged at a side of the measurement section so as not to interfere with the measurement. Information on the inclination position (line-of-sight) of the eyeball obtained by the inclination detector to detect the inclination position of the eyeball can return to the control unit, and measurement can be performed according to the information on the inclination position of the eyeball.

In an eye test device including the line-of-sight detector according to the embodiments.

Further, the line-of-sight detector 10 can be applied to a user state estimation device that estimates the state of the user based on information about the inclination of the three-dimensional object 30 (the eyeball), the pupil position (cornea), or the line-of-sight direction. Herein, the user represents a user of the user state estimation device.

The state of the user includes at least one of the degree of fatigue of the user or the attention level of the user. The degree of the fatigue of the user is, for example, an indicator representing the degree of mental fatigue of the user. The attention level of the user is an indicator representing the level of attention of the user.

In a user state estimation device including the line-of-sight detector according to the embodiments; and an estimation unit to estimate a state of the user based on the line-of-sight of the eyeball.

In the user state estimation device according to the embodiments, the state of the user includes at least one of a degree of fatigue of the user or an attention level of the user.

For example, a user state estimation device to estimate the degree of fatigue of a user includes a line-of-sight detector 10 and a fatigue estimation unit to estimate the degree of fatigue based on the line-of-sight direction information of the user detected by the line-of-sight detector 10. The fatigue estimation unit is an example of the state estimation unit.

There is an estimation method of the mental fatigue of the user in the fatigue estimation unit. For example, the estimation method is described in NPL 2. According to the method, the mental fatigue can be estimated by performing a task of tracking the trajectory of an object displayed on a monitor with the eyes for a few minutes and measuring the movements of the eyes during the task. The line-of-sight detector 10 can detect light reflected from an object with higher sensitivity while preventing the amount of light emitted to the object (e.g., an eyeball) from increasing. Thus, the fatigue estimation device including the line-of-sight detector 10 can estimate the mental fatigue of the user safely and with higher accuracy. Further, the fatigue estimation device may include notification means to notify the user of information to urge the user, for example, to take a break, based on the estimated mental fatigue.

The user state estimation device to estimate the attention level of a user includes a line-of-sight detector 10 and an attention level estimation unit to estimate the attention level of the user on the basis of the line-of-sight direction information detected by the line-of-sight detector 10. The attention level estimation unit is an example of the state estimating unit.

As an estimation method of the attention level of the user in the attention level estimation unit, for example, there is a method of detecting a minute vibration of the three-dimensional object 30 (the eyeball) referred to as a microsaccade and estimating the attention level of the user based on the frequency of occurrence of the vibration. The microsaccade is a high-speed motion with relatively large amplitude among microtremors of fixation (i.e., microvibrations of the eyeball with an amplitude of ±3.0° that occur when a person is gazing at an object), and has a correlation with the attention of a person. For example, the microsaccade is described in NPL 3. Since the line-of-sight detector 10 can measure the inclination of the three-dimensional object 30 (the eyeball) at higher speed and with higher accuracy, the line-of-sight detector 10 can detect the microsaccade with higher accuracy as compared with the typical line-of-sight detector.

In the user state estimation device according to the embodiments, the estimation unit estimates the state of the user based on a frequency of occurrence of a minute vibration of the eyeball of the user.

Thus, the user state estimation device to estimate the attention level of the user can detect the reflected light by the object with higher sensitivity while preventing the amount of light irradiating the object from increasing, so that the attention level of the user can be estimated safely and with higher accuracy.

Further, the user state estimation device including the line-of-sight detector 10 can be applied to a driving support system. The driving support system includes a user state estimation device including the line-of-sight detector 10, and an operation control unit to control the operation of a mobile body based on the attention level estimated by the user state estimation device. For example, when the attention level of the user estimated by the user state estimation device is lower than a predetermined standard, the operation control unit controls the operation mode of the mobile body, for example, the vehicle, so as to switch from the manual operation mode to the automatic operation mode. Since the line-of-sight detector 10 can detect the reflected light from an object with higher sensitivity while preventing the amount of light irradiating the object from increasing, the driving support system can perform driving support safely with higher accuracy.

In a driving support system including: the user state estimation device according to the embodiments; and circuitry to control a mobile body driven by the user based on the state of the user estimated by the user state estimation device.

Further, in these embodiments, each of two or more image generation units and a state estimation unit to estimate the state of the user may use one piece of information about the inclination of the three-dimensional object 30 (the eyeball), the pupil position (cornea), or the line-of-sight direction detected by the line-of-sight detector 10. According to the configuration, the reflected light by the eyeballs 30 of the user with higher sensitivity while preventing the amount of light irradiating to the three-dimensional object 30 (the eyeball) of the user from increasing, and the size of line-of-sight detector 10 is reduced.

For example, the inclination of the eyeballs 30, the pupil position (corneas), or the line-of-sight direction information detected by the line-of-sight detector 10 may be used as a feedback signal to the image generation unit of the retinal projection display device, and may be used for fatigue estimation by the fatigue estimation unit of the fatigue estimation device. At this time, the image generation unit and the fatigue estimation unit as the functional configuration unit may be provided in the same information processing device or in separate information processing devices.

In addition, the numbers such as ordinal numbers and quantities used above are all examples for specifically describing the technology of the present invention, and the present invention is not limited to the exemplified numbers. In addition, a connection relation between the components is an example for specifically describing the technology of the present invention, and a connection relation for implementing a function of the present invention is not limited thereto.

The functions of the embodiments described above can be achieved by one or multiple circuits. Herein, the “circuitry”, “processing circuitry”, or “processing circuit” includes a processor programmed to perform each function by software such as a processor implemented by an electronic circuit, and a device such as an application specific integrated circuit (ASIC), a digital signal processor (DSP), a field programmable gate array (FPGA), or a typical circuit module designed to perform each function described above.

Aspects of the present invention are as follows.

In a first aspect, an inclination detector includes: a first support including: a first surface; and a second surface opposite to the first surface; a light source on the first surface, the light source to emit a light beam; a light guide facing the light source, in which the light guide guides the light beam emitted from the light source; a light exit portion to emit the light beam through the first support, in which the light beam guided from the light source on the first surface toward the second surface through the light guide; a first light receiver on the second surface of the first support, in which the first light receiver receives the light beam emitted from the light exit portion and reflected from a three-dimensional object and outputs a first reception signal; and circuitry to detect an inclination of the three-dimensional object based on the first reception signal output from the first light receiver.

In a second aspect, in the inclination detector according to the first aspect, the first support includes a circuit board mounting the light source on the first surface and mounting the first light receiver on the second surface.

In a third aspect, in the inclination detector according to the first aspect or the second aspect, the light source includes multiple light emitting portions.

In a fourth aspect, in the inclination detector according to any one of the first aspect to the third aspect, the light source includes multiple light emitting portions each of which are spaced from each other.

In a fifth aspect, the inclination detector according to any one of the first aspect to the fourth aspect, further includes a light separator adjacent to the light guide, the light separator to separate the light beam emitted from the light source into two or more light beams; and a second light receiver on the first surface, the second light receiver to receive at least one light beam among the two or more light beams and outputs a second reception signal based on the at least one light beam.

In a sixth aspect, the inclination detector according to the fifth aspect, further includes: a light deflector to deflect the light beam emitted from the light source; and multiple second light receivers including the second light receiver, the multiple second light receivers separated from each other. The light source includes multiple light emitting portions spaced from each other, the multiple light emitting portions respectively emit light beams, the light deflector deflects the light beams respectively emitted from the multiple light emitting portions, the multiple second light receivers are respectively paired with the multiple light emitting portion, the second light receiver and the light emitting portion disposed alternately along the first surface, and the light deflector deflects the light beams respectively emitted from the multiple light emitting portions to the multiple second light receivers respectively paired with the multiple light emitting portions.

In a seventh aspect, in the inclination detector according to the sixth aspect, the light source includes: a first light-source substrate; a second light-source substrate; the multiple light emitting portions between the first light-source substrate and the second light-source substrate; a cathode terminal on the first light-source substrate; and an anode terminal on the first light-source substrate. The cathode terminal and one of the multiple light emitting portions are between the first light-source substrate and the second light-source substrate to electrically connect the first light-source substrate and the second light-source substrate, and the cathode terminal and another of the multiple light emitting portions are between the first light-source substrate and the second light-source substrate to electrically connect the first light-source substrate and the second light-source substrate.

In an eighth aspect, the inclination detector according to any one of the first aspect to the seventh aspect, further includes: a second support having a third surface and a fourth surface opposite to the third surface, the light guide on the third surface; and a spacer between the first surface of the first support and the fourth surface of the second support.

In a ninth aspect, in the inclination detector according to any one of the fifth aspect to the eighth aspect, the light guide includes a first light guide and a second light guide on the third surface of the second support, the light separator is on the third surface of the second support and is between the first light guide and the second light guide, each of the first support and the second support has a through hole as the light exit portion, the light beam passed through the first light guide, the light separator, and the second light guide passes through the through hole of each of the first support and the second support.

In a tenth aspect, the inclination detector according to the first aspect to the ninth aspect, further includes: a holder to hold the first support; a reflective light condenser held by the holder, the reflective light condenser to reflect the light beam emitted from the light exit portion to the three-dimensional object while condensing the light beam; a third light receiver to receive the light beam reflected from the reflective light condenser; and circuitry to detect the inclination of the three-dimensional object. The light source includes: a first light emitting portion; and a second light emitting portion, the first light receiver receives the light beam emitted from the first light emitting portion and reflected from the three-dimensional object to output the first reception signal, the third light receiver receives the light beam emitted from the second light emitting portion and reflected from the reflective light condenser to output a third reception signal, and the circuitry detects the inclination of the three-dimensional object, in which a position and an angle of the reflective light condenser are compensated based on each of the first reception signal and the third reception signal. In an eleventh aspect, in a line-of-sight detector including the inclination detector according to any one of the first aspect to the ninth aspect, the three-dimensional object includes an eyeball, and the line-of-sight detector detects a line-of-sight of the eyeball.

In a twelfth aspect, in a head-mounted display including the line-of-sight detector according to the eleventh aspect.

In a thirteenth aspect, in a retinal projection display including the line-of-sight detector according to the eleventh aspect.

In a fourteenth aspect, in an eye test device including the line-of-sight detector according to the eleventh aspect.

In a fifteenth aspect, in a user state estimation device including the line-of-sight detector according to the eleventh aspect; and an estimation unit to estimate a state of the user based on the line-of-sight of the eyeball.

In a sixteenth aspect, in the user state estimation device according to the fifteenth aspect, the estimation unit estimates the state of the user based on a frequency of occurrence of a minute vibration of the eyeball of the user.

In a seventeenth aspect, in the user state estimation device according to the fifteenth aspect or the sixteenth aspect, the state of the user includes at least one of a degree of fatigue of the user or an attention level of the user.

In an eighteenth aspect, in a driving support system including: the user state estimation device according to any one of the fifteenth aspect to the seventeenth aspect; and circuitry to control a mobile body driven by the user based on the state of the user estimated by the user state estimation device.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention. Any one of the above-described operations may be performed in various other ways, for example, in an order different from the one described above.

The present invention can be implemented in any convenient form, for example using dedicated hardware, or a mixture of dedicated hardware and software. The present invention may be implemented as computer software implemented by one or more networked processing apparatuses. The processing apparatuses include any suitably programmed apparatuses such as a general purpose computer, a personal digital assistant, a Wireless Application Protocol (WAP) or third-generation (3G)-compliant mobile telephone, and so on. Since the present invention can be implemented as software, each and every aspect of the present invention thus encompasses computer software implementable on a programmable device. The computer software can be provided to the programmable device using any conventional carrier medium (carrier means). The carrier medium includes a transient carrier medium such as an electrical, optical, microwave, acoustic or radio frequency signal carrying the computer code. An example of such a transient medium is a Transmission Control Protocol/Internet Protocol (TCP/IP) signal carrying computer code over an IP network, such as the Internet. The carrier medium also includes a storage medium for storing processor readable code such as a floppy disk, a hard disk, a compact disc read-only memory (CD-ROM), a magnetic tape device, or a solid state memory device.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), a digital signal processor (DSP), a field programmable gate array (FPGA), and conventional circuit components arranged to perform the recited functions.

This patent application is based on and claims priority to Japanese Patent Application No. 2022-044644, filed on Mar. 18, 2022, in the Japan Patent Office, the entire disclosure of which is hereby incorporated by reference herein.

### REFERENCE SIGNS LIST

1 First support (First substrate)
1a First surface
1b Second surface
2 Second support (Second substrate)
3 Spacer
10, 10a, 10b Line-of-sight detector (an example of inclination detection device)
11, 11a Light source
12, 12a Second light receiver
13 Light exit portion
14 First light receiver
15 Amplifier
16 Connector
17 Flexible substrate
20 Light guide
21 First light guide (First prism)
22 Second light guide (Second prism)
23 First through hole
24 Light separator (Beam splitter)
25 Second through-hole
26 Third through hole
30 A three-dimensional object (Eyeball)
31 Pupil
32 Cornea
33 Retina
40, 45 Light emitting portion
41 Electrode
41p Anode terminal
41n Cathode terminal
42, 42p, 42n Electric conductor
43 First light-source substrate (First semiconductor substrate)
44 Second light-source substrate (Second semiconductor substrate)
50 Optical unit
60 Reflective light condenser
61 RGB laser light source
62 Scanning mirror
63 Plane mirror
64 Half Mirror
65 Image generation unit
70 Eyeglasses support
71 Lens
72 Eyeglasses frame
73 Temple
74 Hinge
80 VCSEL array (Light emitting portions)
81 Air layer
91 Third light receiver
100, 100a Circuitry
101 CPU
102 ROM
103 RAM
104 SSD
105 Light source driving circuit
106 A/D conversion circuit
107 Input/Output Interface (I/F)
108 System Bus
110 Light emission driving unit
111 Signal input unit
114 Light deflector (Polarization anisotropic element)
120 Calculation unit
121 Estimation unit (Eye rotation angle estimation unit)
122 Line-of-sight information acquisition unit
130 Line-of-sight information output unit
200 Retinal projection display
Ct Control signal
Dr drive signal
E Line of sight information
Fd Feedback signal
S Reception signal
M Light amount monitoring signal
L0, L4 Laser light beam
L1, L5 Conversing light beam
L2 Reflected light beam
L3 First order diffraction light beam

1. An inclination detector comprising:
a first support including:
a first surface; and
a second surface opposite to the first surface;
a light source on the first surface, the light source to emit a light beam;
a light guide to guide the light beam emitted from the light source;
a light exit portion to emit the light beam, guided through the light guide, from the first surface toward the second surface; and
a first light receiver on the second surface of the first support, the first light receiver to receive the light beam emitted from the light exit portion and reflected from a three-dimensional object and output a first reception signal.

2. The inclination detector according to claim 1, wherein:
the first support includes a circuit board mounting the light source on the first surface and mounting the first light receiver on the second surface.

3. The inclination detector according to claim 1, wherein:
the light source includes multiple light emitters.

4. The inclination detector according to claim 1, wherein:
the light source includes multiple light emitters spaced from each other.

5. The inclination detector according to claim 1, further comprising:
a light separator adjacent to the light guide, the light separator to separate the light beam emitted from the light source into two or more light beams; and
a second light receiver on the first surface, the second light receiver to receive at least one light beam among the two or more light beams and outputs a second reception signal based on the at least one light beam.

6. The inclination detector according to claim 5, further comprising:
a light deflector to deflect the light beam emitted from the light source; and
multiple second light receivers including the second light receiver, the multiple second light receivers separated from each other,
wherein:
the light source includes multiple light emitters spaced from each other, the multiple light emitters respectively emit light beams,
the light deflector deflects the light beams respectively emitted from the multiple light emitters,
the multiple second light receivers are respectively paired with the multiple light emitters, the second light receiver and the light emitters disposed alternately along the first surface, and
the light deflector deflects the light beams respectively emitted from the multiple light emitters to the multiple second light receivers respectively paired with the multiple light emitters.

7. The inclination detector according to claim 6, wherein:
the light source includes:
a first light-source substrate;
a second light-source substrate;
the multiple light emitters between the first light-source substrate and the second light source substrate;
a cathode terminal on the first light-source substrate; and
an anode terminal on the first light-source substrate,
the cathode terminal and one of the multiple light emitters are between the first light-source substrate and the second light-source substrate to electrically connect the first light-source substrate and the second light-source substrate, and
the cathode terminal and another of the multiple light emitters are between the first light-source substrate and the second light-source substrate to electrically connect the first light-source substrate and the second light-source substrate.

8. The inclination detector according to claim 1, further comprising:
a second support having a third surface and a fourth surface opposite to the third surface, the light guide on the third surface; and
a spacer between the first surface of the first support and the fourth surface of the second support.

9. The inclination detector according to claim 8, wherein:
the light guide includes a first light guide and a second light guide on the third surface of the second support,
the light separator is on the third surface of the second support and is between the first light guide and the second light guide,
each of the first support and the second support has a through hole as the light exit portion, and
the light beam passed through the first light guide, the light separator, and the second light guide passes through the through hole of each of the first support and the second support.

10. The inclination detector according to claim 1, further comprising:
a holder to hold the first support;
a reflective light condenser held by the holder, the reflective light condenser to reflect the light beam emitted from the light exit portion to the three-dimensional object while condensing the light beam;
a third light receiver to receive the light beam reflected from the reflective light condenser; and
circuitry to detect an inclination of the three-dimensional object,
wherein:
the light source includes:
a first light emitter; and
a second light emitter,
the first light receiver receives the light beam emitted from the first light emitter and reflected from the three-dimensional object to output the first reception signal,
the third light receiver receives the light beam emitted from the second light emitter and reflected from the reflective light condenser to output a third reception signal, and
the circuitry detects the inclination of the three-dimensional object, in which a position and an angle of the reflective light condenser are compensated based on each of the first reception signal and the third reception signal.

11. A line-of-sight detector comprising the inclination detector according to claim 1, wherein:
the three-dimensional object includes an eyeball, and
the line-of-sight detector detects a line-of-sight of the eyeball.

12. A head-mounted display comprising the line-of-sight detector according to claim 11.

13. A retinal projection display comprising the line-of-sight detector according to claim 11.

14. An eye test device comprising the line-of-sight detector according to claim 11.

15. A user state estimation device comprising:
the line-of-sight detector according to claim 11; and
estimation circuitry configured to estimate a state of the user based on the line-of-sight of the eyeball.

16. The user state estimation device according to claim 15,
wherein the estimation circuitry estimates the state of the user based on a frequency of occurrence of a minute vibration of the eyeball of the user.

17. The user state estimation device according to claim 15, wherein:
the state of the user includes at least one of a degree of fatigue of the user or an attention level of the user.

18. A driving support system comprising:
the user state estimation device according to claim 15; and
circuitry configured to control a mobile body driven by the user based on the state of the user estimated by the user state estimation device.
